(19) United States
(12) Patent Application Publication
(10) Pub. No.: US 2005/0147660 A1
(43) Pub. Date: Jul. 7, 2005

(54) Peptide-carrying bodies for immune response

(76) Inventors: Anthony E. Bolton, Bakewell (GB); Arkady Mandel, North York (CA)

Correspondence Address: FOLEY & LARDNER LLP, 1530 PAGE MILL ROAD, PALO ALTO, CA, 94304, US

(21) Appl. No.: 10/502,149

(22) PCT Filed: Jan. 21, 2003
(86) PCT No.: PCT/CA03/00066

(30) Foreign Application Priority Data
Jan. 21, 2002 (CA) 2,368,656

(51) Int. Cl. A61K 9/127; A61K 38/06

(52) U.S. Cl. 424/450; 514/18

(57) ABSTRACT

A composition of matter capable of producing an anti-inflammatory response in vivo in a mammal comprises bodies having a three-dimensional core structure of conformation and size from about 20 nm to about 500 μm and expressing or expressible on the surface thereof RGDS ligands which will react, optionally in the presence of adapter molecules, with at least one specific receptor wherein the binding of said ligand with said receptors produces an anti-inflammatory response in vivo in said mammal.

### FIELD OF THE INVENTION

This invention relates to medical and pharmaceutical compositions and medical treatments More specifically, the invention relates to compositions which, on administration to mammalian patients, exert beneficial effects on a patient [symbol] s immune system.

### BACKGROUND OF THE INVENTION

During the normal physiological processes occurring during the life of a mammalian body, cells that become senescent die by a process of programmed cell death also called apoptosis. These dying cells are removed from the body, generally by some type of antigen presenting cell, often to be replaced by cells newly produced by cell division. This is part of the normal cell turnover in the mammalian body. Unlike cells that die by necrotic cell death as a result of a pathological process, such as trauma or infection, cells dying by apoptosis do not elicit an inflammatory response. Indeed, it has recently been demonstrated that cells undergoing apoptosis can exert an actively anti-inflammatory response on the immune system in that they can induce a down-regulation of certain inflammatory cytokines and/or up-regulation of certain anti-inflammatory cytokines (Fadok, Valerie A. et. al., Nature, Vol. 405, 4 May 2000, p85; Scott, Rona S. et. al., Nature, Vol. 411, 10 May 2001, p207.

In the process of apoptosis, the dying cells undergo a change in morphology and in the expression of various ligands present on the outer surface of the cell membrane. These changes in cell surface ligand expression are thought to signal to those cells of the body that remove apoptotic cells. A number of specific ligands expressed on apoptotic cells have been observed to Induce an anti-inflammatory response as a consequence of interaction with receptors, in antigen presenting cells, for example by Inducing the down-regulation of certain inflammatory cytokines and/or the up-regulation of certain anti-inflammatory cytokines by antigen presenting cells. There are a number of cell surface ligands which are present either uniquely or at increased levels on apoptotic cells compared to normal cells. These include phosphatidylserine (PS), a phospholipid normally restricted to the inside of the cell membrane but which becomes transferred to the outside of the membrane during apoptosis, and interacts with PS receptors on antigen presenting cells.

The result of the process of interaction of ligands and receptors in the process of apoptotic death of cells in the mammalian body is a change In the cytokine production profile of various cells in the mammalian immune system, especially the antigen presenting cells involved in the uptake of the products of apoptosis.

Peptides containing the integrin recognition motif RGDS (Arg-Gly-Asp-Ser) are known to interact with receptors on antigen-presenting cells.

U.S. Pat. No. 5,840,691 Furcht et at discloses polypeptide compositions with fibronectin activity, including polypeptides having an RGD or RGDS sequence, for treatment of medical conditions associated with inflammatory conditions. The compositions may be conjugates such as lipids carrying the polypeptide sequences, and are proposed to be used at dosages, for adult humans, of from 1-100 milligrams of polypeptide formulation per kilogram body weight per day. Lestini B. J. et al.: “Surface modification of liposomes for selective cell targeting in cardiovascular drug delivery”, Journal of Controlled Release, Elsevier Science Publisher B. V. Amsterdam, Vol. 78 No. 1-3, pages 235-247 discloses treatment of cardiovascular disease processes, including inflammation, with liposomes conjugated with RGD peptide sequences. In a reported in vivo experiment, 0.75 μmol of total lipid was injected into a 20-30 g mouse, equivalent to about 600 micrograms of lipid.

European patent application EP-A-0298820 Laboratoire LaFon et al. discloses derivative of peptides having the RGDS sequence and their use as fibrinogen antagonists with respect to blood platelate aggregation. They can be prepared by sequential addition to a solid support. In an in vivo experiment, dosages of 0.75-3.0 mg of peptide were administered to mice, with an overall suggested dosage range of 1-500 mg.

Patent Abstracts of Japan. JP 08 183740 Nippon Steel Chem Co. Ltd, discloses RGDS peptide sequences for use as anti-inflammatory pharmaceutical agents.

Ito, Masafumi et al. “T cell adherence and mucosal injury in ulcerative colitis: involvement of integrin-fibronectin interaction in situ”. Journal of Gastroenterology Japan November 1995 Vol. 30 Supplement 8. November 1985, pages 70-72. discloses a study of peripheral blood T-lymphocytes and their increased adhesion to inflamed mucosa from patients with ulcerative colitis. The study reported partial Inhibition of the adherence by antibodies specific for plasma fibronectin, which has an RGD site to which certain Integrins (cell adhesion mediators), bind. The authors suggest that synthetic peptides should be tested, based on these results.

### SUMMARY OF THE INVENTION

The present invention is based on the discovery that the interaction of one or more receptors on antigen presenting cells with the peptide motif sequence RGD alters the cytokine production profile of the antigen presenting cells and/or other cells capable of cytokine production in vivo. The present invention proceeds from this discovery, and comprises the therapeutic application of compositions of matter containing surface RGD motifs that are recognized by one or more of the antigen presenting cell receptors. Such motifs will interact with receptors on antigen presenting cells and perhaps other cells to promote an anti-inflammatory response. The invention comprises the novel compositions of matter, their processes of preparation, their therapeutically useful forms, combinations and compositions, and their therapeutic uses. As a result of the administration of these compositions of matter, an inflammatory autoimmune, cardiovascular and/or neurodegenerative disorder in a mammalian patient is treated or inhibited. It is postulated that, upon interaction with a specific receptor or receptors on cells of the recipient mammalian patient, the cytokine profile of the antigen presenting cells of the mammalian patient is altered by upregulation of one or more anti-inflammatory cytokines and/or down-regulation of one or more inflammatory cytokines. This induces, among other effects, a shift in the balance of the T-cells of the recipient patient's body such that there is a relative increase in regulatory T-cells such as Th-2, Th-3, Tr-1 and/or other regulatory cell populations, and/or a relative decrease in pro-inflammatory T-cells such as Th-1 cells. In this way, the immune system of the recipient mammalian patient is modulated, altering the cytokine profile towards a less inflammatory or an anti-inflammatory profile, in a manner towards alleviation or inhibition of the specific disorder under treatment.

Thus according to one aspect of the present invention, there is provided use in the preparation of a medicament for alleviating or inhibiting the symptoms of inflammation in a mammalian patent of synethetic bodies selected from liposomes, solid beads, hollow beads and filled beads, capable of being phagocytosed in vivo by mammalian antigen-presenting cells resulting in the alteration of the cytokine profile of cells of the mammalian immune system, having a size from about 20 nanometers to 500 microns in diametric dimension, and expressing or having expressible on the surface thereof an active group containing the peptide sequence RGD.

### THE PREFERRED EMBODIMENTS

The preferred peptide motif RGD sequence for use in the present invention is RGDS, and so the invention will be fully described with reference to this sequence.

A composition of matter comprising bodies having a three-dimensional core structure as the term is used herein, refers to a biocompatible composition of matter having a three-dimensional body portion of shapes resembling mammalian cells, typically but not exclusively spheroidal, cylindrical, ellipsoidal including oblate and prolate spheroidal, serpentine, reniform, etc., and sizes from about 20 nanometers (nm) to about 500 micrometers (μm) in diametric dimension. They have the RGDS motif presented on the exterior surface in a manner for interaction with appropriate receptor(s), preferably other than exclusively the PS receptor, on professional or other antigen-presenting cells in vivo.

Examples of three-dimensional body portions include liposomes, solid beads, hollow beads, and filled beads. Synthetic body portions such as liposomes and beads can be prepared synthetically to have the required ligand on their surfaces.

In the process of using the compositions of the invention to alleviate or inhibit inflammation in a mammalian body, the compositions are introduced into the body by suitable means, and then it is believed that the bodies are recognized by antigen-presenting cells and interact therewith through the reaction of the RGDS groups on the body surfaces with specific receptor(s) for the ligands on the antigen-presenting cells, followed in most cases by engulfment and digestion of the bodies by the antigen-presenting cells, in a manner resembling the process of phagocytosis. At some stage in the process, the cytokine profile of the involved cells, most probably the antigen-presenting cells, changes in a direction favoring anti-inflammation. The present invention is not dependent upon any particular theory or mode of action, only on the fact that an anti-inflammatory response is obtained at some stage in the in vivo process following the appropriate administration of the bodies to the patient.

Examples of PS receptors are disclosed in Fadok, V., et. al., International patent application publication WO-01/66785, published 13 Sep., 2001.

More than one receptor may be involved in interaction with RGDS on the bodies according to the present invention, to result in an anti-inflammatory response. The present invention extends to cover this situation, including situations where one of the pluralities of involved receptors is the PS receptor.

In the present invention, the bodies are acting as modifiers of the patient [symbol] s immune system, in a manner somewhat similar to that of a vaccine. Accordingly, they are used in quantities and by administration methods to provide a sufficient localized concentration of the bodies at the site of introduction to initiate the appropriate immune response. Quantities of RGDS-carrying bodies appropriate for immune system modifying substances are generally not directly correlated with body size of the recipient and can, therefore, be clearly distinguished from drug dosages, which are designed to provide therapeutic levels of active substances in the patient's blood stream and tissues. Drug dosages are accordingly likely to be much larger than immune system modifying dosages.

Preferred RGDS-carrying bodies for use in the invention are beads or liposomes of the appropriate size and biocompatibility, with beads being particularly preferred.

Methods of preparing liposomes of the appropriate size are known in the art and do not form part of this invention. Reference may be made to various textbooks and literature articles on the subject, for example, the review article [symbol] Liposomes as Pharmaceutical Dosage Forms [symbol] , by Yechezkel Barenholz and Daan J. A. Chrommelin, and literature cited therein, for example New, R. C. [symbol] Liposomes: A Practical Approach [symbol] , IRL Press at Oxford University Press (1990). The RGDS can be obtained commercially, as a chemical entity. Chemical methods of coupling the RGDS motif to the liposome are easily devised by those skilled in the art, from knowledge of the chemical surface groups on the liposomes available to participate in the chemical coupling reaction.

The diameter of the ligand-carrying liposomes of the preferred embodiment of this invention is from about 20 nm to about 1000 nm, more preferably from about 50 nm to about 500 nm.

The term “beads” used herein in reference to RGDS-carrying bodies in the present invention includes particles, granules, microspheres and beads of biocompatible materials, natural or synthetic, such as polyethylene glycol, polyvinylprrolidone, polystyrene, etc., polysaccharides such as hydroxethyl starch hydroxyethylcellulose, agarose and the like, as commonly used in the pharmaceutical industry. Some such suitable substances for derivatization to attach the ligand are commercially available, e.g. from Polysciences, Inc. 400 Valley Road, Warrington, PA 18976, or from Sigma Aldrich Fine Chemicals. The beads may be solid or hollow, or filled with biocompatible material. They are modified as required so that they carry RGDS active groups on their surfaces. Preferred bead sizes are from about 20 nanometers to about 1000 nanometers, more preferably from about 50-500 nanometers.

The RGDS-carrying bodies may be administered to the patient by any suitable means, which brings them into operative contact with active components of the patient's immune system.

The RGDS-carrying bodies may be suspended in a pharmaceutically acceptable carrier, such as physiological sterile saline, sterile water, pyrogen-free water, isotonic saline, and phosphate buffer solutions, as well as other non-toxic compatible substances used in pharmaceutical formulations. Preferably, the RGDS-carrying bodies are constituted into a liquid suspension in a biocompatible liquid such as buffered saline and administered to the patient in any appropriate route which introduces it to the immune system, such as intra-arterially, intravenously or most preferably intramuscularly or subcutaneously.

It is contemplated that the RGDS-carrying bodies may be freeze-dried or lyophilized so that they may be later re-suspended for administration. This invention is also directed to a kit of part comprising lyophilized or freeze-dried RGDS-carrying bodies and a pharmaceutically acceptable carrier, such as physiological sterile saline, sterile water, pyrogen-free water, isotonic saline, and phosphate buffer solutions, as well as other non-toxic compatible substances used in pharmaceutical formulations.

A preferred manner of administering the RGDS-carrying bodies to the patient is a course of injections, administered daily, several times per week, weekly or monthly to the patient, over a period ranging from a week to several months. The frequency and duration of the course of the administration is likely to vary from patient to patient, and according to the condition being treated, its severity, and whether the treatment is intended as prophylactic, therapeutic or curative. Its design and optimization is well within the skill of the attending physician.

The quantities of RGDS-carrying bodies to be administered will vary depending on the nature of the mammalian disorder it is intended to treat and on the identity and characteristics of the patient. It is important that the effective amount of RGDS-carrying bodies is non-toxic to the patient, and is not so large as to overwhelm the immune system. When using intra-arterial, intravenous, subcutaneous or intramuscular administration of a liquid suspension of RGDS-carrying bodies, it is preferred to administer, for each dose, from about 0.1-50 ml of liquid, containing an amount of RGDS-carrying bodies generally equivalent to 10%. 1000% of the number of leukocytes normally found in an equivalent volume of whole blood. Generally, the number of RGDS active group-carrying bodies administered per delivery to a human patient is in the range from about 500 to about 2.5×10⁹ (<250 ng of bodies, in the case of liposomes, pro-rated for density differences for other embodiments of bodies, e.g. from about 50 to about 5000 ng in the case of solid beads), more preferably from about 10,000 to about 50,000,000, and most preferably from about 200,000 to about 10,000,000.

Since the RGDS-carrying bodies are acting, in the process of the invention, as immune system modifiers, in the nature of a vaccine, the number of such bodies administered to an injection site for each administration is a more meaningful quantitation than the number or weight of RGDS-carrying bodies per unit of patient body weight. For the same reason, it is now contemplated that effective amounts or numbers of RGDS-carrying bodies for small animal use may not directly translate into effective amounts for larger mammals (i.e. greater than 5 Kg) on a weight ratio basis.

The present invention is indicated for use in prophylaxis and/or treatment of a wide variety of mammalian disorders where T-cell function, inflammation, endothelial dysfunction and inappropriate cytokine expression are involved. A patient having or suspected of having such a disorder may be selected for treatment. “Treatment” refers to administration to a patient for purposes of achieving a reduction of symptoms, such as, but not limited to, a decrease in the severity or number of symptoms of the particular disease or to limit further progression of symptoms.

With respect to T-cell function (T-cell mediated) disorders, these may be autoimmune disorders including, but not limited to diabetes, scleroderma, psoriasis and rheumatoid arthritis.

The invention is indicated for use with inflammatory allergic reactions, organ and cell transplantation reaction disorders, and microbial infections giving rise to inflammatory reactions. It is also indicated for use in prophylaxis against oxidative stress and/or ischemia reperfusion injury, ingestion of poisons, exposure to toxic chemicals, radiation damage, and exposure to airborne and water-borne irritant substances, etc., which cause damaging inflammation. It is also indicated for inflammatory, allergic and T-cell-mediated disorders of internal organs such as kidney, liver, heart, etc.

With respect to disorders involving inappropriate cytokine expression for which the present invention is indicated, these include neurodegenerative (neuroinflammatory) diseases. Neurodegenerative diseases, including Down's syndrome, Alzheimer's disease and Parkinson's disease, are associated with increased levels of certain cytokines, including interleukin-1β (IL-1β) (see Griffin WST et al. (1989); Mogi M. et al. (1996)). It has also been shown that 1β inhibits long-term potentiation in the hippocampus (Murray, C. A. et al. (1998)). Long-term potentiation in the hippocampus is a form of synaptic plasticity and is generally considered to be an appropriate model for memory and learning (Bliss, T. V. P. et al. (1993)). Thus, inappropriate cytokine expression in the brain is currently believed to be involved in the development and progression of neurodegenerative and neuroinflammatory diseases.

Thus, the invention is indicated for the treatment and prophylaxis of a wide variety of mammalian neurological disorders, including. Downs syndrome, Alzheimer's disease, Parkinson's disease, senile dementia, depression, Huntingdon's disease, peripheral neuropathies, Guillain Barr syndrome, spinal cord diseases, neuropathic joint diseases, chronic inflammatory demyelinating disease, neuropathies including mononeuropathy, polyneuropathy, symmetrical distal sensory neuropathy, neuromuscular junction disorders, myasthenias and amyotrophic lateral sclerosis (ALS). Treatment and prophylaxis of these neurodegenerative diseases represents a particularly preferred embodiment of the invention, with treatment of Alzheimers and Parkinson [symbol] s disease particularly preferred.

Regarding disorders involving endothelial dysfunction, the present invention is indicated for the treatment and prophylaxis of a wide variety of such mammalian disorders including, but not limited to, cardiovascular diseases, such as atherosclerosis, peripheral arterial or arterial occlusive disease, congestive heart failure, cerebrovascular disease (stroke), myocardial infarction, angina, hypertension, etc., vasospastic disorders such as Raynaud's disease, cardiac syndrome X, migraine etc., and the damage resulting from ischemia (ischemic injury or ischemia-reperfusion injury). In summary, it can be substantially any disorder the pathology of which involves an inappropriately functioning endothelium.

The invention is further described in the following specific examples.

### EXAMPLE 1

Amino-terminal-biotinylated peptide (b-RGDS from Alto Biosciences, UK), and streptavidin coated Dynabeads (M−280, 2.8 μm diameter from Dynal, Norway) were prepared according to methodology contained: in Adderley S R, Fitzgerald D J, J. Biol. Chem. 2000 Feb. 25; 275(8): 5760-6. Briefly, this involves coating the streptavidin coated Dynabeads with biotinylated peptides, according to the manufacturer's instructions. For this, the Dynabeads were re-suspended by vortexing for 2 min., and the required volume was pipetted into a suitable tube, which was placed in the Dynal magnetic protein purification holder and allowed to settle for 2 min. The supernatant was removed carefully, and the beads were re-suspended in PBS. The appropriate amount of biotinylated peptides (1 μg of peptide to 10⁷ beads) was added to washed Dynabeads and incubated for 30 min. at 4° C. with unidirectional mixing. The beads were collected, the supernatant removed, and the beads re-suspended. Washing was repeated six times. The suggested concentration of peptide added to the beads was 1 μg of peptide to 10⁷ beads so that sufficient concentration of peptide (67 ug) was added to 6.7×10⁸ beads as a stock concentration.

### EXAMPLE 2

A stock suspension the RGDS-beads prepared as described in example 1, containing 6.7×10⁸ beads per ml was diluted with PBS to give an injection suspension containing 6×10⁶ beads per ml. The bead suspensions were injected into female BALB/c mice (Jackson Laboratories) aged 6-8 weeks and weighing 19-23 g, to determine the effect on ear swelling in the murine contact hypersensitivity (CHS) model. The CHS model tests for Th1-mediated inflammatory reactions.

The animals were assigned to one of 2 groups, with 5 animals in each group. Group A was a control group, receiving PBS instead while B received approximately 6×10⁵ of the RGDS-beads.

Protocol

The following experiments were performed:

TABLE I

| | | | | | | | Day 7 |
|---|---|---|---|---|---|---|---|
| Group | Day 1 | Day 2 | Day 3 | Day 4 | Day 5 | Day 6 | (24 hours) |
| A | Injected then | Injected | Injected | Injected | Injected | Injected | Ear measured |
| | sensitized | | | | | then | |
| | | | | | | challenged | |
| B | Injected then | Injected | Injected | Injected | Injected | Injected | Ear measured |
| | Sensitized | | | | | then | |
| | | | | | | challenged | |

On Days 1-6, mice of Groups A and B were injected as indicated above. Approximately 600,000 beads were injected in a 50 μl volume via intramuscular (IM) injection, for a total administration over the test period of about 3,600,000 beads.

Sensitization

On Day 1, following liposome injection for that day, mice were anaesthetized with 0.2 ml 5 mg/ml sodium pentobarbital via IP injection. The abdominal skin of the mouse was sprayed with 70% EtOH and a scalpal blade was used to remove about a one-inch diameter patch of hair from the abdomen. The shaved area was then painted with 25 μl of 0.5% 2,4-dinitrofluorobenzene (DNFB) in 4:1 acetone:olive oil using a pipette tip.

Challenge

Following liposome injection on day 6, mice were challenged with DNFB by painting 10 μl of 0.2% DNFB on the dorsal surface of the right ear with a pipette tip and by painting 10 μl of vehicle on the left ear with a pipette tip.

Results

On Day 7, 24 hours after challenge, each animal was anaesthetized with. Halothane, and ear thickness was measured using a Peacock spring-loaded micrometer. Data was expressed as the difference between the treated right ear thickness and the thickness of the vehicle-treated left ear. The experiments were repeated three times, on similar animals. Increase in ear swelling was used as a measure of CHS response. The significance of the data was determined by the two-tailed student's t-test. A P value of <0.05 was considered significant.

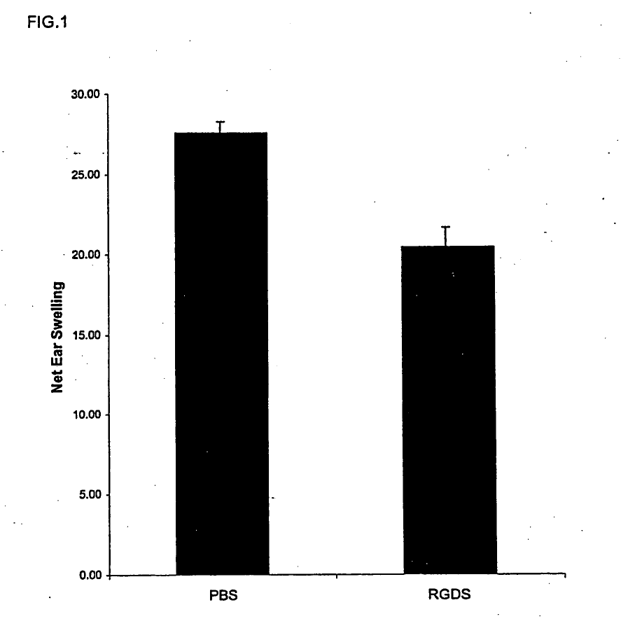

The results are presented in FIG. 1, a bar graph showing the mean values from the three experiments of ear swelling, reported in μm. FIG. 1 shows that a significant reduction in ear swelling was achieved by injection of the RGDS-beads according to the present invention.

### EXAMPLE 3

A stock suspension of RGDS-beads containing 6.7×10⁸ beads per ml was diluted to give an injection suspension containing 6×10⁶ beads per ml. The bead suspensions were used to inject into mice, to determine the effect on ear swelling in the murine Delayed Type Hypersensitivity (DHS) model. As in example 1, female BALB/c mice (Jackson Laboratories) aged 6-8 weeks and weighing 19-23 g were used.

The animals were assigned to one of 2 groups, 10 animals in each group. One group received the bead injections. The other was a control group that received PBS injections. Each test animal was injected with 50 μl of suspension containing 6×10⁵ beads.

Protocol

Mice were sensitized on day 1, challenged on day 6, challenged a second time on day 12, and injected on days 13, 14, 15, 16, 17 and 18 with the beads. On day 18, after the bead injections, the mice were challenged. Beads were injected in a 50 μl volume via IM injection, i.e. 600,000 beads per injection, for a total administration over the test period of 3,600,000 beads. Sensitization and challenge took place as described in Example 1. Ear thickness was measured on day 19.

Results

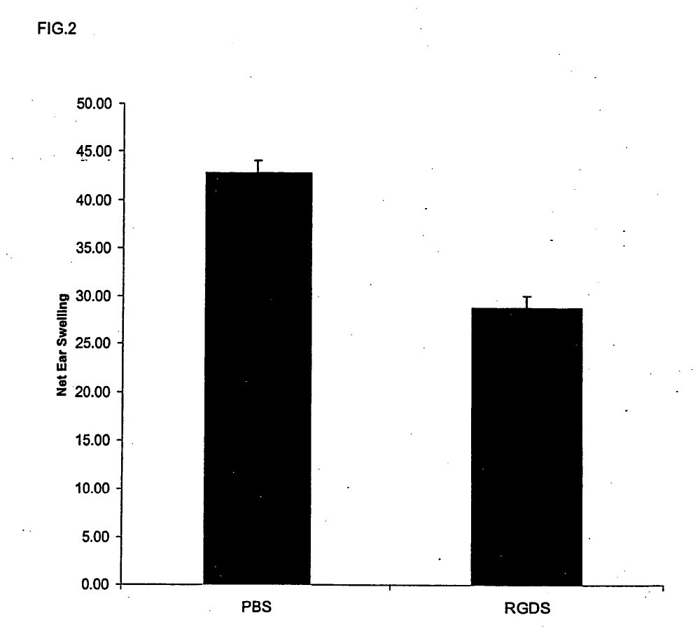

The results are presented in bar graph form on accompanying FIG. 2 where the results are expressed as Net Ear Swelling (×10⁻² m). They show that RGDS-beads are effective in the DHS model on day 19, 24 hours after the 3^{rd} injection following the third challenge.

1. A composition of matter comprising bodies selected from the group consisting of liposomes, solid beads, hollow beads and filled beads, further wherein the bodies have a size from about 20 nanometers to 500 microns in diametric dimension, still further wherein the bodies contain or are capable of expressing or expressible on the surface thereof an active group comprising the peptide sequence RGD, wherein the bodies are capable of being phagocytosed in vivo in a mammal by mammalian antigen-presenting cells resulting in the alteration of the cytokine profile of cells of the mammalian immune system and thereby producing an anti-inflammatory response in vivo in said mammal.

2. The composition of matter according to claim 1, wherein the bodies contain on their surface thereof an active group comprising the peptide sequence RGD.

3. The composition of matter according to claim 2 wherein the active group is the peptide sequence RGDS.

4-12. (canceled)

13. A method of alleviating or inhibiting the symptoms of inflammation in a mammalian patient, which comprises administering to the patient an effective amount of a composition of matter comprising synthetic bodies having a three-dimensional core structure of size from 20 nanometers to 500 microns expressing or expressible on the surface thereof RGDS ligands which will react, optionally in the presence of adapter molecules, with at least one specific receptor wherein the binding of said ligand with said receptors produces an anti-inflammatory response in vivo in said mammal.

14. The method according to claim 13, wherein the symptoms of inflammation are caused by a neurodegenerative disorder.

15. The method according to claim 14, wherein the neurodegenerative disorder is Parkinson's disease.

16. The method according to claim 14, wherein the neurodegenerative disorder is Alzheimer's disease.

17. The method according to claim 13, wherein the symptoms of inflammation are caused by a cardiovascular disease.

18. The method according to claim 17, wherein the cardiovascular disease is atherosclerosis.

19. The method according to claim 13, wherein the symptoms of inflammation are caused by an autoimmune disease.

20. The method according to claim 13, wherein the symptoms of inflammation are caused by endothelial dysfunction.
